(19) United States
(12) Patent Application Publication
(10) Pub. No.: US 2021/0379578 A1
(43) Pub. Date: Dec. 9, 2021

(54) APPARATUS WITH A SENSOR HAVING AN ACTIVE SURFACE

(71) Applicant: ILLUMINA, INC., San Diego, CA (US)

(72) Inventors: Wesley Cox-Muranami, Middleton, WI (US); Rajagopal Panchapakesan, Escondido, CA (US); Arnaud Rival, Saint Nazaire les Eymes (FR)

(21) Appl. No.: 17/324,363

(22) Filed: May 19, 2021

Related U.S. Application Data
(60) Provisional application No. 62/704,963, filed on Jun. 4, 2020.

(51) Int. Cl. B01L 3/00; B01L 7/00; C12Q 1/6869

(52) U.S. Cl. CPC B01L 3/502; B01L 7/00; C12Q 1/6869; B01L 2300/168; B01L 2200/026; B01L 2300/046; B01L 2300/042; B01L 2300/0663; B01L 2200/10; B01L 2300/1827

(57) ABSTRACT

An apparatus and examples of methods for using and manufacturing aspects of an apparatus with a sensor having an active surface. A sensor, a lid, and a flow channel bounded by the lid and a surface of the sensor, and including an illumination source, a heater, and a pump. A method includes fluidically coupling a first flow cell and a second flow cell to a reservoir, moving fluid from the reservoir into a flow channel of the first and second flow cell using respective pumps; and heating fluid in the flow channels of the first and second flow cells using respective heaters. A method includes forming a first sensor and a second sensor on a flexible surface, and folding the flexible surface until the first sensor faces the second sensor.

### RELATED APPLICATION SECTION

This application claims the benefit of and priority to U.S. Provisional Patent Application No. 62/704,963, filed Jun. 4, 2020, the content of which is incorporated by reference herein in its entirety and for all purposes.

### BACKGROUND

Various protocols in biological or chemical research involve performing controlled reactions. The designated reactions can then be observed or detected and subsequent analysis can help identify or reveal properties of chemicals involved in the reaction. In some multiplex assays, an unknown analyte having an identifiable label (e.g., fluorescent label) can be exposed to thousands of known probes under controlled conditions. Each known probe can be deposited into a corresponding well of a microplate. Observing any chemical reactions that occur between the known probes and the unknown analyte within the wells can help identify or reveal properties of the analyte. Other examples of such protocols include known deoxyribonucleic acid (DNA) sequencing processes, such as sequencing-by-synthesis (SBS) or cyclic-array sequencing.

In some fluorescent-detection protocols, an optical system is used to direct excitation light onto fluorophores, e.g., fluorescently-labeled analytes and to also detect the fluorescent emissions signal light that can emit from the analytes having attached fluorophores. In other proposed detection systems, the controlled reactions in a flow cell are detected by a solid-state light sensor array (e.g., a complementary metal oxide semiconductor (CMOS) detector). These systems do not involve a large optical assembly to detect the fluorescent emissions. For CMOS based flow cells that use external illumination, the lid over the flow channel may be transparent. Furthermore, the external illumination source often is aligned with the sensor, which may have particular challenges for removable flow cells and/or multiple flow cells used in a single instrument. External illumination may also result in shadows caused by inlets in the lid for some flow cells.

Some sequencing, such as DNA sequencing, may include moving reagents, buffers, and/or other materials through a flow channel over a sensor, such as a CMOS sensor, maintaining and/or modifying the temperature(s) of the materials within the flow channel, and illuminating fluorescent nucleotides within the flow channel. To use a shared pool of reagent resources for each flow cell may involve a fluidic solution that passes fluids to multiple flow cells on demand.

### SUMMARY

Accordingly, it may be beneficial for individually addressable CMOS flow cells to enable a user to load multiple sequestered samples into a single sequencing run without the need for additional reagent cartridges, using shared reagent volumes and random accessibility. Sequencing instruments may use shared hardware components among several samples on individual addressable flow cells rather than a 1-to-1 regime. Shared hardware components may allow for higher sequencing output without a significant increase in the corresponding cost of an instrument. Individually addressable flow cells may provide ‘random access’ functionality on sequencers as the individually addressable flow cells can be added or subtracted at any point in time during a sequencing run, thereby allowing for multiple sequencing runs to start and stop at the same or different times, and even during the middle of a particular sequencing run without affecting the sequencing runs of other individually addressable flow cells. Users may load smaller sample volumes into flow cells and multiplexing flow cells rather than multiplexing sample input, thereby reducing the need of an excessive amount of sample input on large output flow cells for factory style platforms. Such implementations may be particularly useful and beneficial for assays that produce a much smaller input concentration (PCR-free assays, for example) that still translate to a factory scale need in terms of the sample variety that is sequenced.

At least some of the examples of the flow cells described herein help enable ‘random access’ sequencing and shared individual control of multiple flow cells on a single instrument. Shared vats or reservoirs of sequencing reagents are accessed on demand by loaded flow cells which can start and stop at any time depending on the type of sequencing run that is programmed for that specific flow cell. The flow cell may include an individual sensor, such as a CMOS type imaging sensor, heating elements, and an electrically controllable pump. Each flow cell may be completely electrically addressable and may individually drive its own imaging, heating, and fluidic pumping.

Thus, shortcomings of the prior art can be overcome and benefits as described later in this disclosure can be achieved through the provision of an apparatus for use in a sensor system or instrument. Various examples of the apparatus are described below, and the apparatus, including and excluding the additional examples enumerated below, in any combination (provided these combinations are not inconsistent), may overcome these shortcomings and achieve the benefits described herein. One example apparatus comprises a sensor with an active surface having a plurality of reaction sites, a lid, and a flow channel formed at least partially by the active surface of the sensor and the lid, where the lid comprises an illumination source.

In some examples of the apparatus, the sensor comprises a Complementary Metal-Oxide Semiconductor (CMOS) detection device.

In some examples of the apparatus, the CMOS detection device comprises a plurality of detection pixels.

In some examples of the apparatus, the lid further comprises a non-transparent material.

In some examples of the apparatus, the lid further comprises an opaque material.

In some examples of the apparatus, the lid further comprises a fluidic channel therein, where the fluidic channel is in fluidic communication with the flow channel.

In some examples of the apparatus, the lid further comprises a reservoir.

In some examples of the apparatus, the reservoir comprises a reagent.

In some examples of the apparatus, the reservoir comprises a buffer.

In some examples of the apparatus, the lid further comprises a heater.

In some examples of the apparatus, the heater is a resistive heater.

In some examples of the apparatus, the lid is on an opposite side of the flow channel from the active surface of the sensor.

In some examples of the apparatus, the illumination source comprises a light emitting diode (LED).

In some examples of the apparatus, the illumination source comprises a plurality of LEDs.

In some examples of the apparatus, the illumination source is located along a periphery of the lid.

In some examples of the apparatus, the lid may also comprise a plurality of light guides, whereby the light guides are to guide light from the illumination source toward the active surface of the sensor.

In some examples of the apparatus, the illumination source comprises a thin film organic LED.

In some examples of the apparatus, the illumination source comprises a silicon-based LED.

In some examples of the apparatus, the illumination source is on a bottom surface of the lid, where the bottom surface of the lid faces the active surface of the sensor.

In some examples of the apparatus, the apparatus further comprises a pump, where the pump is fluidically coupled to the flow channel. The pump may be downstream from the sensor.

In some examples of the apparatus, the lid further comprises an outlet port, wherein the pump is adjacent to the outlet port of the lid.

In some examples of the apparatus, there is no removable connection between the flow channel and the pump.

In some examples of the apparatus, the pump is a piezoelectric pump having a flexible diaphragm element.

Shortcomings of the prior art can be overcome and benefits as described later in this disclosure can be achieved through the provision of a method of performing a biological or chemical analysis. Various examples of a method are described below, and the method, including and excluding the additional examples enumerated below, in any combination (provided these combinations are not inconsistent), overcome these shortcomings and achieve the benefits described herein. One example method comprises fluidically coupling a first flow cell and a second flow cell to a reservoir, wherein the first flow cell and second flow cell each comprise a sensor with an active surface having a plurality of reaction sites, a lid, a heater, and a pump, where the lid and the sensor at least partially form a flow channel, where the pump is in fluidic communication with the flow channel; moving fluid from the reservoir into the flow channel of the first flow cell using the pump of the first flow cell and fluid from the reservoir into the flow channel of the second flow cell using the pump of the second flow cell; and heating fluid in the flow channel of the first flow cell using the heater of the first flow cell such that fluid in the flow channel of the first flow cell is at a different temperature than the fluid in the flow channel of the second flow cell.

In some examples of the method, moving fluid from the reservoir into the flow channel of the first flow cell does not occur while moving fluid from the reservoir into the flow channel of the second flow cell.

In some examples of the method, the reservoir comprises a reagent.

In some examples of the method, the reservoir comprises a buffer.

In some examples of the method, the method further comprises illuminating at least a portion of the reaction sites of the sensor of the first flow cell.

In some examples of the method, the method further comprises illuminating at least a portion of the reaction sites of the sensor of the second flow cell.

In some examples of the method, illuminating at least a portion of the reaction sites of the sensor of the second flow cell does not occur while illuminating at least a portion of the reaction sites of the sensor of the first flow cell.

In some examples of the method, an illumination source in the lid of the first flow cell illuminates at least a portion of the reaction sites of the sensor of the first flow cell.

In some examples of the method, an illumination source in the lid of the second flow cell illuminates at least a portion of the reaction sites of the sensor of the second flow cell.

In some examples of the method, a first sequencing run is performed on the first flow cell, and a second sequencing run is performed on the second flow cell, where the first sequencing run and second sequencing run start at different times.

Shortcomings of the prior art can be overcome and benefits as described later in this disclosure can be achieved through the provision an apparatus for use in a sensor system or instrument. Various examples of the apparatus are described below, and the apparatus, including and excluding the additional examples enumerated below, in any combination (provided these combinations are not inconsistent), overcome these shortcomings and achieve the benefits described herein. One example apparatus comprises a sensor with an active surface having a plurality of reaction sites, a lid, and a flow channel formed at least partially by the active surface of the sensor and the lid, where the lid comprises a heater.

In some examples of the apparatus, the heater is a resistive heater.

In some examples of the apparatus, the sensor comprises a Complementary Metal-Oxide Semiconductor (CMOS) detection device.

In some examples of the apparatus, the CMOS detection device comprises a plurality of detection pixels.

In some examples of the apparatus, the apparatus further comprises a pump, where the pump is fluidically coupled to the flow channel.

In some examples of the apparatus, the pump is downstream from the sensor.

In some examples of the apparatus, the lid further comprises an outlet port, wherein the pump is adjacent to the outlet port of the lid.

In some examples of the apparatus, there is no removable connection between the flow channel and the pump.

In some examples of the apparatus, the pump is a piezoelectric pump having a flexible diaphragm element.

Shortcomings of the prior art can be overcome and benefits as described later in this disclosure can be achieved through the provision of an apparatus for use in a sensor system or instrument. Various examples of the apparatus are described below, and the apparatus, including and excluding the additional examples enumerated below, in any combination (provided these combinations are not inconsistent), overcome these shortcomings and achieve the benefits described herein. One example apparatus comprises a first sensor and a second sensor, where each of the first and second sensors comprise an active surface having a plurality of reaction sites, where the active surface comprises a plurality of embedded illumination sources, where a flow channel is formed at least partially by the active surface of the first sensor and the active surface of the second sensor, where the active surface of the first sensor faces the active surface of the second sensor.

In some examples of the apparatus, the embedded illumination sources are embedded into spaces between the reaction sites of the active surface of each of the first sensor and second sensor.

In some examples of the apparatus, each of the embedded illumination sources is a light emitting diode (LED).

In some examples of the apparatus, the apparatus further comprises a pump, where the pump is fluidically coupled to the flow channel.

In some examples of the apparatus, the pump is downstream from the flow channel.

In some examples of the apparatus, there is no removable connection between the flow channel and the pump.

In some examples of the apparatus, the pump is a piezoelectric pump having a flexible diaphragm element.

Shortcomings of the prior art can be overcome and benefits as described later in this disclosure can be achieved through the provision of a method of making a portion of a flow cell. Various examples of the method are described below, and the method, including and excluding the additional examples enumerated below, in any combination (provided these combinations are not inconsistent), overcome these shortcomings and achieve the benefits described herein. One example method comprises forming a first sensor and a second sensor on a flexible surface, where each of the first and second sensors comprises an active surface having a plurality of reaction sites, where the active surface comprises a plurality of embedded illumination sources; and folding the flexible surface until the first sensor faces the second sensor, whereby a flow channel is formed between the first sensor and second sensor.

In some examples of the method, the illumination sources are embedded into spaces between the reaction sites of the active surface of each of the first sensor and second sensor.

In some examples of the method, each of the embedded illumination sources is a light emitting diode (LED).

In some examples of the method, the method further comprises fluidically coupling a pump to the flow channel.

In some examples of the method, the pump is downstream from the flow channel.

In some examples of the method, there is no removable connection between the flow channel and the pump.

In some examples of the method, the pump is a piezoelectric pump having a flexible diaphragm element.

Additional features are realized through the techniques described herein. Other examples and aspects are described in detail herein and are considered a part of the claimed aspects. These and other objects, features and advantages of this disclosure will become apparent from the following detailed description of the various aspects of the disclosure taken in conjunction with the accompanying drawings.

It should be appreciated that all combinations of the foregoing aspects and additional concepts discussed in greater detail below (provided such concepts are not mutually inconsistent) are contemplated as being part of the inventive subject matter and to achieve the benefits advantages disclosed herein.

### BRIEF DESCRIPTION OF THE DRAWINGS

One or more aspects are particularly pointed out and distinctly claimed as examples in the claims at the conclusion of the specification. The foregoing and objects, features, and advantages of one or more aspects are apparent from the following detailed description taken in conjunction with the accompanying drawings in which:

FIG. 1 depicts a side view of an example of a flow cell that includes a heater and a pump;

FIG. 2 depicts a top view of an example of a flow cell shown in FIG. 1;

FIG. 3 depicts a side view of an example of a flow cell shown in FIG. 1 secured within a receptacle;

FIG. 4 depicts an example of a system with multiple flow cells illuminated by a single light source;

FIG. 5 depicts an example of a system with multiple flow cells fluidically coupled to shared fluidic sources;

FIG. 6 depicts an example of a portion of a flow cell with a lid having an embedded light source;

FIG. 7 depicts an example of a portion of a flow cell with a lid having an embedded light source and heater;

FIG. 8 depicts an example of a portion of a flow cell with a lid having a light source on its outer surface;

FIG. 9 depicts an example of a portion of a flow cell with a lid having a light source on its outer surface and embedded heater;

FIG. 10 depicts an example of a portion of a flow cell with a heater and a lid having a light source on its outer surface;

FIG. 11 depicts an example of a portion of a flow cell with a lid having peripheral light sources and a waveguide;

FIG. 12 depicts an example of a portion of a flow cell with a lid having a thin film organic light emitting diode;

FIG. 13 depicts an example of a portion of a flow cell with a silicon based light emitting diode lid;

FIG. 14 depicts an example of a portion of a flow cell with a sensor in a mold having through-mold vias;

FIG. 15 depicts another example of a portion of a flow cell with a sensor in a mold having through-mold vias;

FIG. 16 depicts an example of a portion of a flow cell with a lid having external pins;

FIG. 17 depicts another example of a portion of a flow cell with a lid having external pins;

FIG. 18 depicts a side view of an example of a portion of a flow cell having a lid with embedded fluidic channels;

FIG. 19 depicts a top view of an example of a portion of a flow cell shown in FIG. 18;

FIG. 20 depicts a bottom schematic view of an example of a portion of a flow cell shown in FIG. 18;

FIG. 21 depicts an example of a portion of a flow cell having a lid with embedded fluidic channels and reservoirs;

FIG. 22 depicts an example of a portion of a flow cell with multiple sensors with a shared lid;

FIG. 23 depicts an example of a sensor with embedded light sources on its active surface;

FIG. 24 depicts an example of a portion of a flow cell with opposing sensors with embedded light sources;

FIG. 25 depicts another example of a portion of a flow cell with opposing sensors with embedded light sources;

FIG. 26 depicts an example of sensors on a flexible surface;

FIG. 27 depicts an example of sensors folded together on a flexible surface;

FIG. 28 depicts a flow chart of a method of operating an instrument with multiple individually addressable flow cells; and

FIG. 29 depicts a flow chart of a method of making a flow cell with opposing sensors.

### DETAILED DESCRIPTION

The accompanying figures, in which like reference numerals refer to identical or functionally similar elements throughout the separate views and which are incorporated in and form a part of the specification, further illustrate the present implementation(s) and, together with the detailed description of the implementation(s), serve to explain the principles of the present implementation(s). As understood by one of skill in the art, the accompanying figures are provided for ease of understanding and illustrate aspects of certain examples of the present implementation(s). The implementation(s) is/are not limited to the examples depicted in the figures.

The terms “connect,” “connected,” “contact” “coupled” and/or the like are broadly defined herein to encompass a variety of divergent arrangements and assembly techniques. These arrangements and techniques include, but are not limited to (1) the direct joining of one component and another component with no intervening components therebetween (i.e., the components are in direct physical contact); and (2) the joining of one component and another component with one or more components therebetween, provided that the one component being “connected to” or “contacting” or “coupled to” the other component is somehow in operative communication (e.g., electrically, fluidly, physically, optically, etc.) with the other component (notwithstanding the presence of one or more additional components therebetween). It is to be understood that some components that are in direct physical contact with one another may or may not be in electrical contact and/or fluid contact with one another. Moreover, two components that are electrically connected, electrically coupled, optically connected, optically coupled, fluidly connected or fluidly coupled may or may not be in direct physical contact, and one or more other components may be positioned therebetween.

The terms “including” and “comprising”, as used herein, mean the same thing.

The terms “substantially”, “approximately”, “about”, “relatively”, or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, they can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. If used herein, the terms “substantially”, “approximately”, “about”, “relatively,” or other such similar terms may also refer to no fluctuations, that is, ±0%.

As used herein, a “flow cell” can include a device having a lid extending over a reaction structure to form a flow channel therebetween that is in communication with a plurality of reaction sites of the reaction structure, and can include a detection device that detects designated reactions that occur at or proximate to the reaction sites. A flow cell can also or alternatively include two (or more) opposing sensors, without a lid. A flow cell may include a solid-state light detection or “imaging” device, such as a Charge-Coupled Device (CCD) or Complementary Metal-Oxide Semiconductor (CMOS) (light) detection device. The CMOS detection device or sensor, for example, may include a plurality of detection pixels that detects incident emission signals. In some examples, each detection pixel corresponds to a reaction site. In other examples, there may be more or fewer pixels than the number of reaction sites. Likewise, a detection pixel in some examples corresponds to a single sensing element to create an output signal. In other examples, a detection pixel corresponds to multiple sensing elements to create an output signal. As one specific example, a flow cell can fluidically, electrically, or both fluidically and electrically couple to a cartridge, which can fluidically, electrically, or both fluidically and electrically couple to a bioassay system. A cartridge and/or bioassay system may deliver a reaction solution to reaction sites of a flow cell according to a predetermined protocol (e.g., sequencing-by-synthesis), and perform a plurality of imaging events. Alternatively, as described herein, the flow cell may contain some or all of the reaction solution for delivery to the reaction sites. For example, a cartridge and/or bioassay system may direct one or more reaction solutions through the flow channel of the flow cell, and thereby along the reaction sites. At least one of the reaction solutions may include four types of nucleotides having the same or different fluorescent labels. In some examples, the nucleotides bind to the reaction sites of the flow cell, such as to corresponding oligonucleotides at the reaction sites. The cartridge, bioassay system, or the flow cell itself in some examples then illuminates the reaction sites using an excitation light source (e.g., solid-state light sources, such as light-emitting diodes (LEDs)). In some examples, the excitation light has a predetermined wavelength or wavelengths, including a range of wavelengths. The fluorescent labels excited by the incident excitation light may provide emission signals (e.g., light of a wavelength or wavelengths that differ from the excitation light and, potentially, each other) that may be detected by the light sensors of the flow cell.

Flow cells described herein perform various biological or chemical processes and/or analysis. More specifically, the flow cells described herein may be used in various processes and systems where it is desired to detect an event, property, quality, or characteristic that is indicative of a designated reaction. For example, flow cells described herein may include or be integrated with light detection devices, sensors, including but not limited to, biosensors, and their components, as well as bioassay systems that operate with sensors, including biosensors.

The flow cells facilitate a plurality of designated reactions that may be detected individually or collectively. The flow cells perform numerous cycles in which the plurality of designated reactions occurs in parallel. For example, the flow cells may be used to sequence a dense array of DNA features through iterative cycles of enzymatic manipulation and light or image detection/acquisition. As such, the flow cells may be in fluidic communication with one or more microfluidic channels that deliver reagents or other reaction components in a reaction solution to a reaction site of the flow cells. The reaction sites may be provided or spaced apart in a predetermined manner, such as in a uniform or repeating pattern. Alternatively, the reaction sites may be randomly distributed. Each of the reaction sites may be associated with one or more light guides and one or more light sensors that detect light from the associated reaction site. In one example, light guides include one or more filters for filtering certain wavelengths of light. The light guides may be, for example, an absorption filter (e.g., an organic absorption filter) such that the filter material absorbs a certain wavelength (or range of wavelengths) and allows at least one predetermined wavelength (or range of wavelengths) to pass therethrough. In some flow cells, the reaction sites may be located in reaction recesses or chambers, which may at least partially compartmentalize the designated reactions therein. Furthermore, the designation reactions may involve or be more easily detected at temperatures other than at ambient temperatures, for example, at elevated temperatures.

As used herein, a “designated reaction” includes a change in at least one of a chemical, electrical, physical, or optical property (or quality) of a chemical or biological substance of interest, such as an analyte-of-interest. In particular flow cells, a designated reaction is a positive binding event, such as incorporation of a fluorescently labeled biomolecule with an analyte-of-interest, for example. More generally, a designated reaction may be a chemical transformation, chemical change, or chemical interaction. A designated reaction may also be a change in electrical properties. In particular flow cells, a designated reaction includes the incorporation of a fluorescently-labeled molecule with an analyte. The analyte may be an oligonucleotide and the fluorescently-labeled molecule may be a nucleotide. A designated reaction may be detected when an excitation light is directed toward the oligonucleotide having the labeled nucleotide, and the fluorophore emits a detectable fluorescent signal. In another example of flow cells, the detected fluorescence is a result of chemiluminescence or bioluminescence. A designated reaction may also increase fluorescence (or Förster) resonance energy transfer (FRET), for example, by bringing a donor fluorophore in proximity to an acceptor fluorophore, decrease FRET by separating donor and acceptor fluorophores, increase fluorescence by separating a quencher from a fluorophore, or decrease fluorescence by co-locating a quencher and fluorophore. A biological or chemical analysis may include detecting a designated reaction.

As used herein, “downstream” refers to being situated in a direction where a net volume of fluid flows towards. For example, if the net flow of fluid flows from a first source, to a second source, such that after a relevant period of time, for example after a DNA sequencing run, more fluid flows from the first source to a second source, the second source is downstream from the first source.

As used herein, “electrically coupled” and “optically coupled” refers to a transfer of electrical energy and light waves, respectively, between any combination of a power source, an electrode, a conductive portion of a substrate, a droplet, a conductive trace, wire, waveguide, nanostructures, other circuit segment and the like. The terms electrically coupled and optically coupled may be utilized in connection with direct or indirect connections and may pass through various intermediaries, such as a fluid intermediary, an air gap and the like Likewise, “fluidically coupled” refers to a transfer of fluid between any combination of sources. The term fluidically coupled may be utilized in connection with direct or indirect connections, and may pass through various intermediaries, such as channels, wells, pools, pumps, and the like.

As used herein, a “reaction solution,” “reaction component” or “reactant” includes any substance that may be used to obtain at least one designated reaction. For example, potential reaction components include reagents, enzymes, samples, other biomolecules, and buffer solutions, for example. The reaction components may be delivered to a reaction site in the flow cells disclosed herein in a solution and/or immobilized at a reaction site. The reaction components may interact directly or indirectly with another substance, such as an analyte-of-interest immobilized at a reaction site of the flow cell.

As used herein, the term “reaction site” is a localized region where at least one designated reaction may occur. A reaction site may include support surfaces of a reaction structure or substrate where a substance may be immobilized thereon. For example, a reaction site may include a surface of a reaction structure (which may be positioned in a channel of a flow cell) that has a reaction component thereon, such as a colony of nucleic acids thereon. In some flow cells, the nucleic acids in the colony have the same sequence, being for example, clonal copies of a single stranded or double stranded template. However, in some flow cells a reaction site may contain only a single nucleic acid molecule, for example, in a single stranded or double stranded form.

As used herein, the term “transparent” refers to allowing all or substantially all visible and non-visible electromagnetic radiation or light of interest to pass through unobstructed; the term “opaque” refers to reflecting, deflecting, absorbing, or otherwise obstructing all or substantially all visible and non-visible electromagnetic radiation or light of interest from passing through; and the term “non-transparent” refers to allowing some, but not all, visible and non-visible electromagnetic radiation or light of interest to pass through unobstructed.

As used herein, the term “waveguide” refers to a structure that guides waves, such as electromagnetic waves, with minimal loss of energy by restricting the transmission of energy to a particular direction or range of directions.

Reference is made below to the drawings, which are not drawn to scale for ease of understanding, wherein the same reference numbers are used throughout different figures to designate the same or similar components.

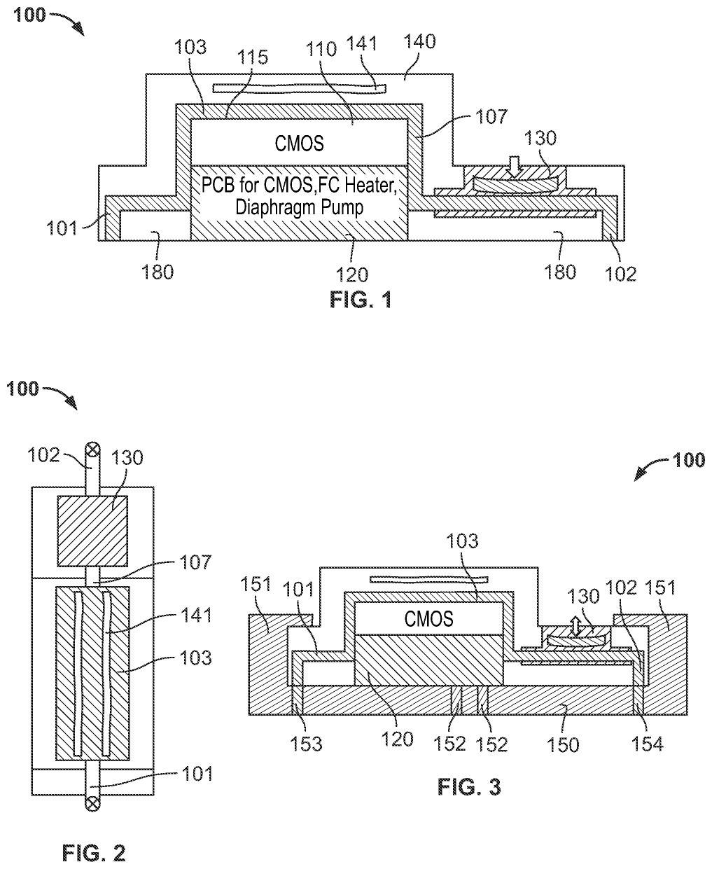

FIG. 1 depicts a side view of an example of a flow cell 100 that includes a heater and a pump. The flow cell 100 includes a sensor 110, for example, an imager sensor such as a CMOS sensor. A top surface of the sensor 110 forms an active surface 115, which may have a plurality of reaction sites. Above the active surface 115 of the sensor 110 is a (micro)-fluidic flow channel 103 delineated by a lid 140 of the flow cell 100 on one side, and a contiguous surface including the active surface 115 of the sensor 110, and optionally, fanout regions extending outward from the active surface 115 of the sensor 110. In other words, the lid 140 bounds at least a portion of the flow channel 103 opposite of the sensor 110. In fabricating a flow cell 100, this fluidic flow channel 103 may be formed over a CMOS or other sensor utilizing one or more of a variety of molding processes, which involve a fabrication technique consisting of multiple processes. If a fluidic flow channel 103 is not formed in a useable shape, reagents may not be exchanged (e.g., single pot reagents) or may not be exchanged in a manner that renders reliable results. Thus, it is desirable that the resultant flow cell 100 include a fluidic flow channel 103 that may be utilized with bio-sensor processes including, but not limited to, SBS or cyclic-array sequencing. Flow channel 103 is fluidically coupled to a fluid inlet 101 and a fluid outlet 102.

The sensor 110 shown in FIG. 1 may be attached to a substrate 120, for example, a printed circuit board (PCB), a ceramic, or other material. Sensor 110 may be attached to the substrate 120 using, for example, a die-attach adhesive paste or film that may provide, for example, low or ultra-low stress on the sensor and high temperature stability. Examples of die-attach pastes include Supreme 3HTND-2DA and EP3HTSDA-1 by MasterBond (USA), and LOCTITE ABLESTIK ATB-F100E by Henkel Corp. USA. An example of a die attach adhesive film is LOCTITE ABLESTIK CDF100 by Henkel Corp. (USA). In one example, the sensor 110 may be directly attached to the substrate 120, while in other examples a structure, coating or layer may be interposed between the substrate 120 and the sensor 110.

In the example shown in FIG. 1, lid 140 includes a heater 141. The heater 141, when activated, provides thermal energy to, among others, the flow channel 103. In some examples, the heater 141 is transparent. A transparent heater in the lid 140 may be important when excitation light is emitted through the lid 140 into the flow channel 103 and onto the active surface 115 of the sensor 110 as a part of a biological or chemical analysis. In other examples, the heater 141 is opaque. An opaque heater may be acceptable when no excitation light is used as a part of a biological or chemical analysis, or when the excitation light used as a part of a biological or chemical analysis is provided to the flow channel 103 and onto the active surface 115 of the sensor 110 without having to travel through the lid 140 of the flow cell 100. In some examples, the heater 141 is a resistive heater.

The flow cell 100 shown in FIG. 1 also includes a pump 130, such as a piezoelectric diaphragm pump. The pump 130 is fluidically coupled to the flow channel 103 via channel 107 as well as fluid outlet 102, and may cause fluid to flow from the fluid inlet 101, through the flow channel 103, and out through the fluid outlet 102. The pump 130 may draw fluid through the flow channel 103, for example, by generating a net negative pressure downstream from the flow channel 103. In some examples, the pump 130 may also cause fluid to travel through the flow channel 103 in the opposite or upstream direction; that is, from the fluid outlet 102 to the fluid inlet 101. In some examples, substrate 120 comprises circuitry to drive the pump 130. In other examples, pump 130 is an electrically controllable pump driven by a controller located externally from the flow cell 100, either through a connection via the substrate 120 or via a separate connection. While a piezoelectric diaphragm pump is shown in the example of FIG. 1, other types of pumps may also be suitable for certain implementations, including without limitation syringe pumps. In some examples, there may be no removable connection between the flow channel 103 and the pump 130. A mold 180 may encapsulate the pump 130, sensor 110, and substrate 120. As shown in FIG. 1, the mold 180 may also form or encapsulate the fluid inlet 101, fluid outlet 102, and channels 107 connecting to the flow channel 103, as well as support or otherwise attach to the lid 140.

The flow cell 100 depicted in FIG. 1 may be an individually addressable CMOS flow cell 100. Each flow cell 100 includes a CMOS imaging surface, piezoelectric pump (or other electrically controllable pump), heating element, fluidic inlet and outlet, and printed circuit board (PCB) to communicate with an electrically coupled instrument. In some examples, the individually accessible flow cell 100 includes a CMOS sensor that is directly embedded into an injection molded plastic body. Using direct adhesives or pressure sensitive adhesives are example methods to fluidically interface the two elements. A piezo pump including a flexible diaphragm element and oscillating electromagnet may drive fluidics. Embedded heating elements, whether they are resistive elements on the CMOS surface itself, transparent resistive elements on the lid 140 of the flow cell 100 (for example, using indium tin oxide), or non-transparent heating elements in the lid 140, may perform directed heating on demand. The plastic body of the assembly may interface directly with an instrument using a pogo pin array for electric communication and at a fluidic inlet and outlet. The heater and pump can be individually controlled, allowing individually addressable sequencing in larger systems or instruments.

FIG. 2 depicts a top view of an example of a flow cell 100 shown in FIG. 1. The flow cell 100 includes a fluid inlet 101 that is fluidically coupled to flow channel 103. A heater 141 is shown over the flow channel 103 as two discreet elements. In other examples, heater 141 may be a single element, or more than two elements. In other examples, heater 141 may be planer, rectangular, oval, linear, circular, or other shape. A channel 107 fluidically couples the flow channel 103 to the pump 130, which in turn is fluidically coupled to the fluid outlet 102.

FIG. 3 depicts a side view of an example of a flow cell 100 shown in FIG. 1 secured within a receptacle 150. A flow cell 100 is shown secured within a receptacle 150. Clasps 151 may be used to removably physically restrain the flow cell 100 within the receptacle 150. Examples of clasps 151 include spring loaded members that rotate about an axis and flexible members with an overhang. In other examples, where magnetic forces do not affect the designated reaction(s) and/or detection or analysis thereof, permanent magnetic or electromagnetic clasps may be used. Electrical connections 152 contact the underside of substrate 120. A fluid source 153 connects to fluid inlet 101 of the flow cell 100. A fluid waste channel 154 connects to fluid outlet 102 of the flow cell 100. In some examples, a pump 130, when activated, draws a fluid from the fluid source 153, through the fluid inlet 101, into the flow channel 103, and then through channel 107, through pump 130, out through fluid outlet 102, and then into fluid waste channel 154. In other examples, a pump 130 may be run in reverse, in which case fluid is drawn back from the fluid waste channel 154, through the fluid outlet 102, and towards the fluid inlet 101. This may be useful, for example, when cyclically moving fluids through the flow channel 103 in forward and reverse directions, that is, creating a backwash flow profile. While the pump 130, which is downstream from the sensor 110 and flow channel 103, may operate in both a forward and reverse direction, in some examples, the net fluid flow is from the fluid source 153 to the fluid waste channel 154. It should be appreciated that multiple different types of fluids may be supplied through the fluid source 153 while being driven by the pump 130. For example, a plurality of reagent wells may be fluidically coupled to a switchable valve, such as a rotary valve, which selectively fluidically couples the fluid source 153 to a particular reagent well. Such selection of the reagent well may be determined by a logic circuit formed on or in substrate 120 alone. In other examples, the reagent well may be determined by an instrument to which the flow cell 100 is secured to within the receptacle 150.

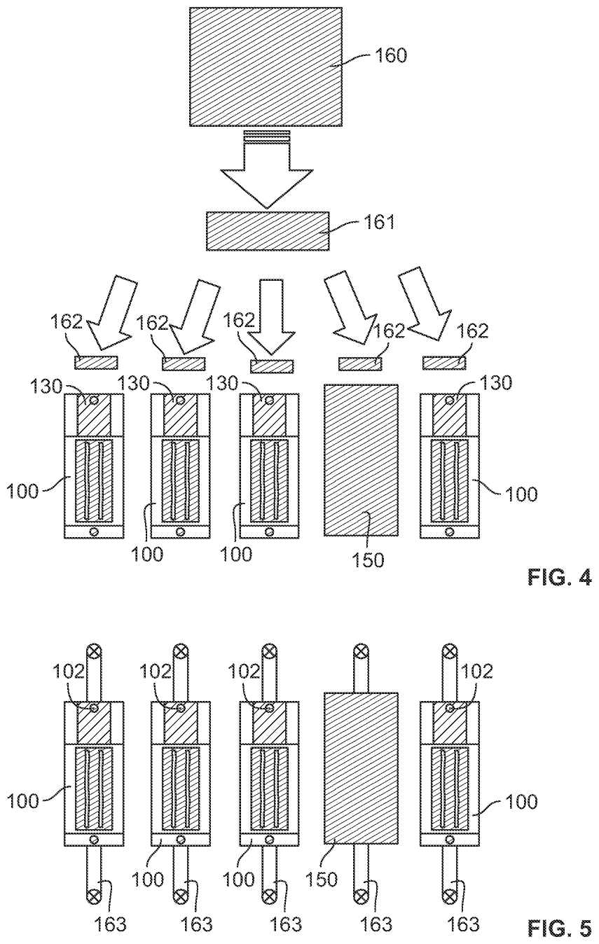

FIG. 4 depicts an example of a system with multiple flow cells 100 illuminated by a single light source 160. A light source 160 emits light, such as excitation light, that travels through a splitter 161. The splitter 161 distributes the excitation light to a plurality of flow cells 100. In this FIG. 4, five receptacles 150 housing four flow cells 100 are shown. It should be appreciated that other examples may include fewer or more than five receptacles, of which some or all of the receptacles may house flow cells, or even no flow cells when not in use. Light gates 162, such as on/off mirrors, corresponding to each receptable, selectively allow excitation light to travel and illuminate the flow cell 100, specifically, the flow channel 103 and reaction sites of the flow cell 100, as a part of the biological or chemical analysis.

In some examples, an instrument interfaces with one or more individually addressable flow cells. Each flow cell resides in an individual nest or receptacle with electronic and fluidic contacts. The reaction sites on the active surface of the sensor of each flow cell is illuminated by either a shared or individualized light source 160, such as a light emitting diode (LED) light source. A light pipe, mirror, or splitter element may enable shared LED source utilization. Self-illuminating flow cells may also be used, such as those described herein, to enable even more compact size and specified addressability of each flow cell 180.

FIG. 5 depicts an example of a system with multiple flow cells 100 fluidically coupled to shared fluidic sources. Similar to FIG. 4, five receptacles 150 housing four flow cells 100 are shown. It should be appreciated that other examples may include fewer or more than five receptacles, of which some or all of the receptacles may house flow cells, or even no flow cells while not in use. In this example, each flow cell 100, when residing within and mated to a receptacle 150, is fluidically coupled to one or more shared fluidic sources, such as sequencing reagents and washes. A valve 163 may select and/or regulate the fluid that is accessible by each flow cell 100. Accordingly, for example, the pump 130 of each flow cell 100 may draw fluid in through the fluid inlet 101, where the specific fluid that is delivered is selected by the valve 163 such as a rotary valve. In other examples, a plurality of valves is utilized to switch between various fluids. Fluid outlet 102 may be fluidically coupled to a waste reservoir. In some examples, the waste reservoir is shared between each of the flow cells 100. In other examples, each flow cell 100 may be fluidically coupled to its own waste reservoir, unshared with others. In yet other examples, each flow cell 100 may be fluidically coupled to its own individual waste reservoir, and each of the individual reservoirs are fluidically coupled to a shared waste reservoir that may be used as an overflow reservoir.

As the individually addressable flow cells 100 may utilize a shared pool of reagent resources rather than carrying their own individual pools, a fluidic solution that can pass fluids to multiple flow cells 100 on demand may be desirable. Having the pump built into the flow cell allows each flow cell 100 to dictate the amount of volume passed over its surface which may be dependent on the overall data output of that specific flow cell 100.

Built in heating of the CMOS flow cell 100 enables each flow cell 100 to be at different points in a sequencing run, even from those adjacent. Sequencing instruments that utilize instrument-based heating usually involve adjacent flow cells 100 addressed at the same temperature and thus each flow cell 100 is aligned on the same sequencing step which is being performed. Built in heating on the flow cell 100 allows for a random access sequencer.

The built-in heating and pumping of the individually addressable flow cells 100 may enable more flexible upstream workflows that may be performed on the flow cells. If, for instance, library preparation and clustering in one instrument and sequencing in the other is desired, the built in functionality of the individually addressable flow cell will alleviate design requirements on each instrument, thereby lowering the overall instrument costs.

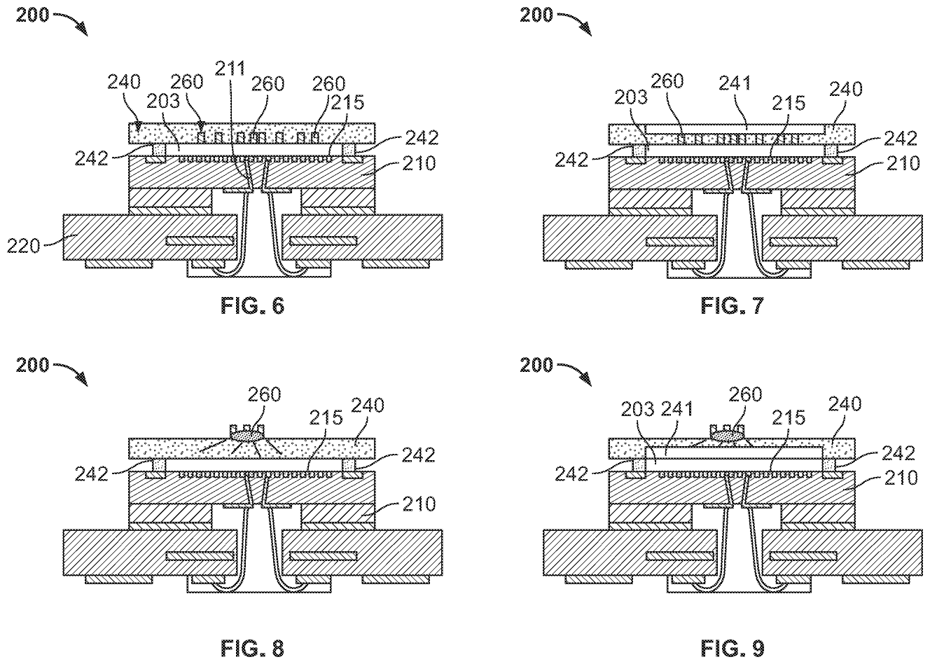

FIG. 6 depicts an example of a portion of a flow cell 200 with a lid 240 having an embedded light source. The flow cell 200 includes a sensor 210 with an active surface 215. The sensor 210 resides over and is coupled to a substrate 220. A lid 240 resides over the active surface 215 of the sensor 210, separated by pillars 242. In other words, the pillars 242 support the lid 240 over the active surface 215 of the sensor 210. For example, an adhesive is applied to the lid 240 and the upper surface of the pillars 242. The adhesive forms an interface between an upper surface of each pillar 242 and the lid 240. In some examples, the pillars 242 are a single continuous material. In other examples, the pillars 242 include multiple layers of materials. In other examples, the pillars 242 comprise multiple components. A flow channel 203 is formed between and bounded by the lid 240 and active surface 215, among other components such as the pillars 242. Light sources 260 are embedded within the lid 240. In some examples, the light sources 260 are light emitting diodes. As shown in this example, the light sources 260 may be unevenly distributed through the lid 240. In other examples, the light sources 260 are evenly distributed through the lid 240, that is, having equal distance spacing between each of the light sources 260. Moreover, as shown in this example, the light sources 260 are located at or on the bottom surface of the lid 240, where the bottom surface of the lid 240 is the surface that is closest to the active surface 215 of the sensor 210. When the light sources 260 are activated, they emit light, such as excitation light, into the flow channel 203. The fluorescent labels excited by the incident excitation light may provide emission signals (e.g., light of a wavelength or wavelengths that differ from the excitation light and, potentially, each other) that may be detected by the sensor 210 of the flow cell 200. In some examples, the lid 240 is opaque. In other examples, the lid 240 is transparent. In some examples, the lid 240 may comprise a transparent glass material. In other examples, the lid 240 may comprise a plastic material that may be opaque.

In some examples, the pillars are a single continuous material. In other examples, the pillars include multiple layers of materials. In other examples, the pillars comprise multiple components. In yet other examples, the pillars are an extension of and continuous with the mold.

FIG. 7 depicts an example of a portion of a flow cell 200 with a lid 240 having an embedded light source and heater. The flow cell 200 in this example is similar to that of FIG. 6, and also includes a heater 241. The heater 241 is embedded within the lid 240, over the light sources 260. The heater 241 may be opaque or otherwise obstruct incident light from outside the flow cell 200. The heater 241, when activated, may provide thermal energy to the flow channel 203, thereby heating the contents therein. The light sources 260, when activated, emit light, such as excitation light, into the flow channel 203.

FIG. 8 depicts an example of a portion of a flow cell 200 with a lid 240 having a light source 260 on its outer surface. Light source 260 in this example is located on the outer surface of the lid 240, that is, the surface that is furthest from the active surface 215 of the sensor 210. The light source 260 may be one or more discreet sources of light, such as one or more light emitting diodes, grouped together. The lid 240 may be optically translucent or otherwise diffusive such that light emitted by the light source 240 is distributed over all or substantially all of the active surface 215 of the sensor 210. In other examples, the light source 260 is a plurality of light sources distributed evenly or unevenly over some or all of the top surface of the lid 240.

FIG. 9 depicts an example of a portion of a flow cell 200 with a lid 240 having a light source on its outer surface and embedded heater 241. The flow cell 200 in this example is similar to that of FIG. 8, and also includes a heater 241 in the lid 240. The heater 241, when activated, may provide thermal energy to the flow channel 203, thereby heating the contents therein. In some examples, the heater 241 is transparent. In other examples, the heater 241 is not transparent, but is of sufficiently small size to not significantly block excitation light from the light source 260. For example, the heater 241 may be a thin resistive heater that allows for light to pass between the heater elements, through the lid 240 toward the active surface 215 of the sensor 210.

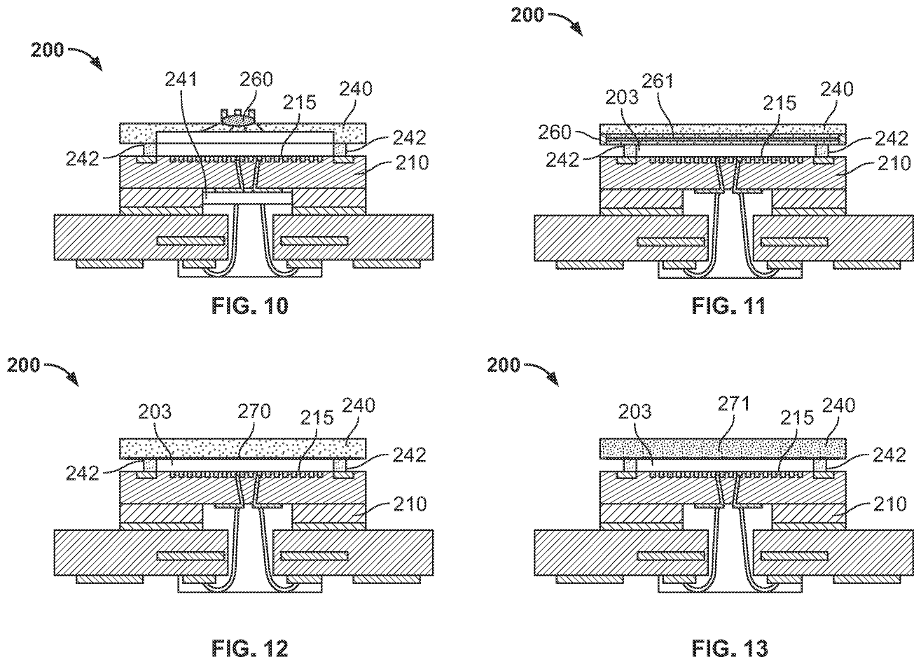

FIG. 10 depicts an example of a portion of a flow cell 200 with a heater 241 and a lid 240 having a light source on its outer surface. The flow cell 200 in this example is similar to that of FIG. 8, and also includes a heater 241 located below the sensor 210. The heater 241, when activated, may provide thermal energy to the flow channel 203, thereby heating the contents therein. The heater 241 may be non-transparent or opaque since it is not located between the light source 260 and the active surface 215 of the sensor 210. However, thermal energy produced by the heater 241 will pass through the sensor 210 to reach the flow channel 203. Such a configuration may be less desirable where the sensor 210 is sensitive to heat, that is, where the sensor's performance becomes degraded at an elevated temperature due to heat from the heater 241 involved for the biological or chemical analysis.

FIG. 11 depicts an example of a portion of a flow cell 200 with a lid 240 having peripheral light sources and a waveguide 261. The lid 240 of the flow cell 200 includes light sources 260 along the periphery of the lid 240. Light emanating from the light sources 260 are directed into the flow channel 203 by a waveguide 261. The waveguide 261 may be a plurality of waveguides that distribute the light produced by the light sources 260 into the flow channel 203. In some examples, the light is evenly distributed or substantially evenly distributed over the active surface 215 of the sensor 210. In some examples, the lid 240 may be opaque. In other examples, the lid 240 may be transparent or non-transparent.

In some examples, the waveguide 261 may comprise more than one layer and one of these additional layers can act as a planarization layer or act as an optical filter. In order to couple light into a waveguide, a grating may be formed that diffracts the light into the propagating direction (modes) of a waveguide. An example of such a waveguide 261 can be a planar waveguide. To achieve high efficiency and high tolerance room (on the angular direction of the light incident on the grating) the size of the coupling structure (e.g., grating) plays a role; it may be designed to be larger.

FIG. 12 depicts an example of a portion of a flow cell 200 with a lid 240 having a thin film organic light emitting diode. The lid 240 has a thin-film organic LED (OLED) 270. The OLED is a thin layer or film that emits light in response to an electric current. The OLED 270 may be located on the bottom surface of the lid 240; that is, the OLED 270 is located on a surface of the lid 240 that is closest to the active surface 215 of the sensor 210. Accordingly, the OLED 270 at least partially bounds the flow channel 203. In other examples, the OLED 270 is located within the lid 240 such that at least a portion of the lid 240 resides between the OLED 270 and the flow channel 203. In other examples, the OLED 270 is located on the top surface of the lid 240.

FIG. 13 depicts an example of a portion of a flow cell 200 with a silicon-based light emitting diode lid. In this example, all or substantially all of the lid 240 of the flow cell 200 comprises a silicon-based LED 271. The silicon-based LED 271 at least partially bounds the flow channel 203 and, when activated, emits light therein toward the active surface 215 of the sensor 210. In other examples, the lid 240 comprises a silicon-based LED 271.

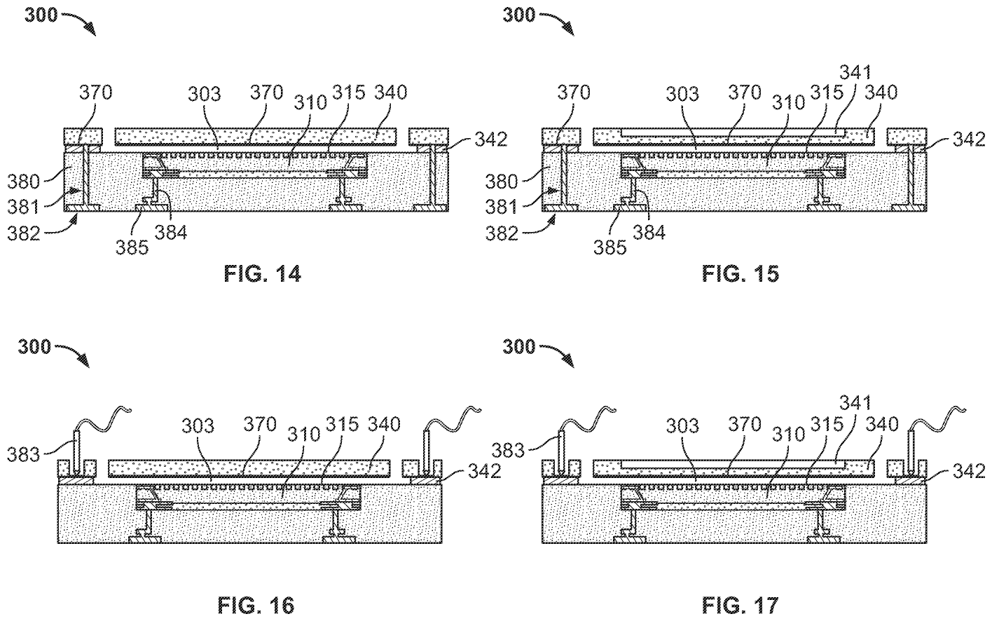

FIG. 14 depicts an example of a portion of a flow cell 300 with a sensor 310 in a mold 380 having through-mold vias. A sensor 310 has an active surface 315 having a plurality of reaction sites thereon. The sensor 310 resides within a mold 380 and is electrically connected to through-mold vias 284 that extend through the mold 380 to pads 385. A lid 340 resides over the active surface 315 of the sensor 310, separated by pillars 342. In other words, the pillars 342 support the lid 340 over the active surface 315 of the sensor 310. In some examples, the pillars 342 are a single continuous material. In other examples, the pillars 342 include multiple layers of materials. In other examples, the pillars 342 comprise multiple components. In yet other examples, the pillars are an extension of and continuous with the mold 380. A flow channel 303 is formed between and bounded by the lid 340 and active surface 315, among other components such as the pillars 342. Through mold vias (TMV) 381 extend from pads 382 on a bottom surface of the mold 380, through the mold 380, through pillars 342 and to the lid 340. In this example, the lid 340 includes a thin film OLED 370 on its bottom surface. The OLED 370 is electrically connected to the TMV 381. Accordingly, current may be provided to the OLED 370 in the lid 340 through the pads 382.

FIG. 15 depicts another example of a portion of a flow cell 300 with a sensor in a mold 380 having through-mold vias 381. The flow cell 300 in this example is similar to that of FIG. 14, and also includes a heater 341 located in the lid 340. The heater 341 may be powered through an electrical connection directly or indirectly to a TMV 381. In some examples, the heater 341 is connected to the same TMV 381 as the light source, such as the OLED 370. In these examples, the OLED 370 and heater 341 are activated or powered together, that is at the same time. In other examples, there are multiple TMVs 381 that extend from separate pads 382 on the bottom of the mold 380 to the lid 340 to selectively provide power to and activate the OLED 370 and heater 341.

FIG. 16 depicts an example of a portion of a flow cell 300 with a lid 340 having external pins 383. A lid 340 resides over the active surface 315 of the sensor 310, separated by pillars 342. In other words, the pillars 342 support the lid 340 over the active surface 315 of the sensor 310. External pins 383 extend through the lid 340 and are electrically connected to the light source in the lid 340, which in this figure is an OLED 370. Accordingly, current may be provided to the OLED 370 in the lid 340 through the pins 383.

FIG. 17 depicts another example of a portion of a flow cell 300 with a lid 340 having external pins. The flow cell 300 in this example is similar to that of FIG. 16, and also includes a heater 341 in the lid 340. The heater 341 may be powered through an electrical connection directly or indirectly to an external pin 383. In some examples, the heater 341 is connected to the same pin 383 as the light source, such as the OLED 370. In these examples, the OLED 370 and heater 341 are activated or powered together, that is at the same time. In other examples, there are multiple pins 383 to selectively provide power to and activate the OLED 370 and heater 341.

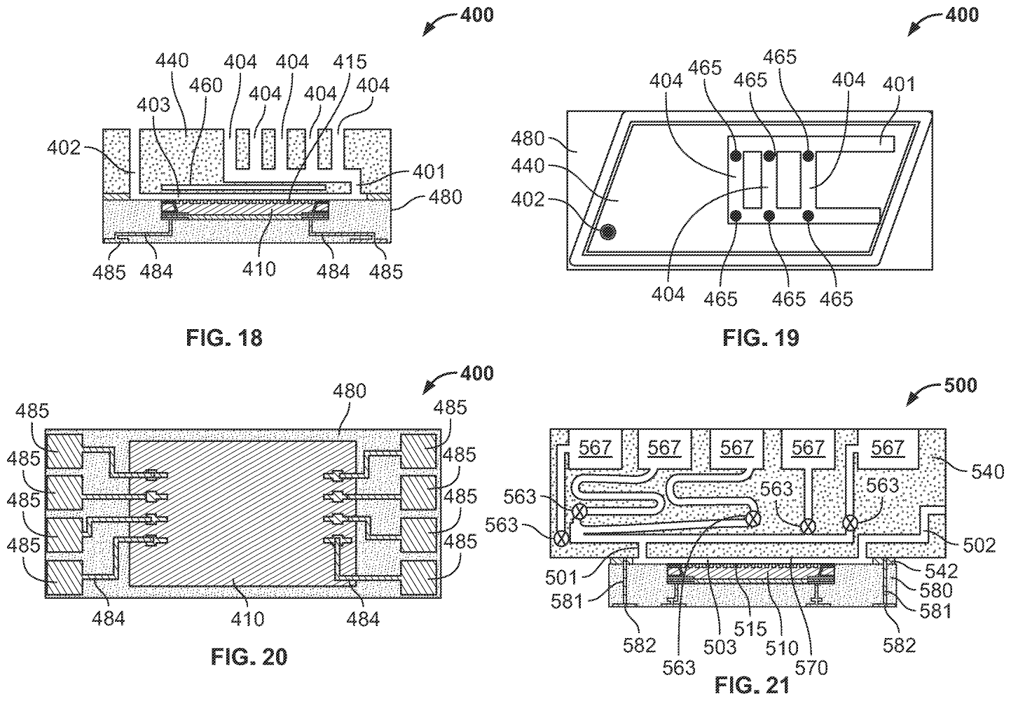

FIG. 18 depicts a side view of an example of a portion of a flow cell 400 having a lid 440 with embedded fluidic channels. A flow cell 400 has a sensor 410 with an active surface 415. The sensor 410 resides in a mold 480 with TMVs 484 extending therethrough to electrically connect the sensor 410 to pads 485. A lid 440 resides over the active surface 415 of the sensor 410, separated by pillars 442. In other words, the pillars 442 support the lid 440 over the active surface 415 of the sensor 410. A flow channel 403 is formed between and bounded by the lid 440 and active surface 415, among other components such as the pillars 442. The lid includes a light source 460. The light source 460 may be on or proximate to the bottom surface of the lid 440, that is, the surface of the lid 440 that is closest to the active surface 415 of the sensor 410. The light source may be a single LED, a plurality of LEDs, LEDs along the periphery of the lid 440 utilizing a waveguide to distribute light on the active surface 415 of the sensor, a thin film OLED, a silicon-based LED, or other light source. Multiple fluid source channels 404 feed into and are fluidically coupled to fluid inlet 401. Fluid inlet 401 is fluidically coupled to flow channel 403, which in turn is fluidically coupled to fluid outlet 402. In operation, fluids, such as reagents and washes, flow through fluid source channels 404, through fluid inlet 401, and into flow channel 403. The fluid then travels out through fluid outlet 402. The fluid may be moved, for example, by a pump, such as those described herein. While the flow of fluid described herein has been described flowing in a downstream direction from fluid inlet 401 to fluid outlet 402, it is nonetheless possible that the flow may travel in an opposite direction. While five fluid source channels are shown in this figure, it should be appreciated that there may be fewer or more than five fluid source channels depending upon the particular implementation.

FIG. 19 depicts a top view of an example of a portion of a flow cell 400 shown in FIG. 18. Flow cell 400 includes a lid 440 over a mold 480 housing a sensor (not shown in this figure). Fluid is provided through inlet ports 465 to channels 404 that connect to fluid inlet 401. Fluid then travels through a flow channel (not shown in this figure) over the sensor, and out through fluid outlet 402.

FIG. 20 depicts a bottom schematic view of an example of a portion of a flow cell 400 shown in FIG. 18. Sensor 410 resides within mold 480. Through mold vias (TMV) 484 connect the sensor 410 to bond pads 485 at the bottom of the mold 480. While eight bond pads are shown in this figure, it should be appreciated that there could be fewer or more than 8 bond pads and connections depending upon the particular implementation.

FIG. 21 depicts an example of a portion of a flow cell 500 having a lid 540 with embedded fluidic channels and reservoirs. A sensor 510 has an active surface 515 having a plurality of reaction sites thereon. The sensor 510 resides within a mold 580 and is electrically connected to through-mold vias 581 that extend through the mold 580 to pads 582. A lid 540 resides over the active surface 515 of the sensor 510, separated by pillars 542. In other words, the pillars 542 support the lid 540 over the active surface 515 of the sensor 510. A flow channel 503 is formed between and bounded by the lid 540 and active surface 515, among other components such as the pillars 542. Through mold vias (TMV) 581 extend from pads 582 on a bottom surface of the mold 580, through the mold 580, through pillars 542 and to the lid 540. In this example, the lid 540 includes a thin film OLED 570 on its bottom surface. The OLED 570 is electrically connected to the TMV 581. Accordingly, current may be provided to the OLED 570 in the lid 540 through the pads 582.

In addition to the OLED 570, ing In certain examples, including the one shown in this FIG. 21, each reservoir may be coupled to the inlet port 501 via a channel and a valve 563 to regulate the flow from each reservoir 567 into the fluid inlet 501. Fluid entering from inlet port 501 travels through flow channel 503 as a part of a biological or chemical analysis. After travelling through the flow channel 503, the fluid exits through the fluid outlet 502. As described in other examples herein, fluid may be drawn through the channels, including the fluid inlet 501, flow channel 503, and out through the fluid outlet 502, by a pump (not shown in this figure).

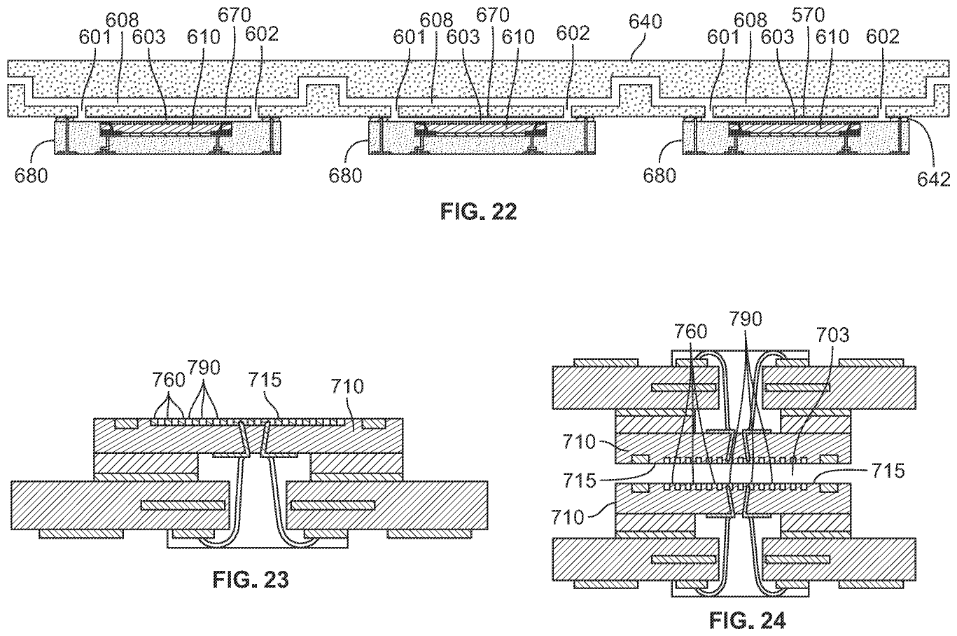

FIG. 22 depicts an example of a portion of a flow cell with multiple sensors 610 with a shared lid 640. A lid 640 is secured to sensors 610, each within a mold 680, via pillars 642. A flow channel 603 is formed between and bounded by the lid 640 and active surface 615 of each sensor 610, among other components such as the pillars 642. Fluid enters the flow channel 603 above each sensor 610 through fluid inlet 601 and exits through fluid outlet 602. Within the lid 640 and above the flow channel 603 resides a bypass channel 608, which provides an alternative route through which fluid may flow through the lid 640, instead of through the flow channel 603 above one of the sensors 610. Fluid flowing through the lid 640, may either travel through the bypass channel 608 or into the fluid inlet 601 and into the corresponding flow channel 603. Fluid flowing through the flow channel 603 exits through fluid outlet 602 and joins fluid flowing through bypass channel 608 and into transfer channel 609 towards the next fluid inlet 601 and bypass channel 608 of the next sensor 610. After fluid exits the fluid outlet 602 and bypass channel 608 of the last sensor 601, the fluid exits lid 640.

The fluidic paths depicted in FIG. 22 show each flow channel 603 in series with another flow channel 603. In other examples, the flow channels 603 may be arranged in parallel, that is, where fluid travelling through a flow channel 603 or bypass channel 608 of one sensor 610 does not flow through a flow channel 603 or bypass channel 608 of another sensor 610. In some examples, some but not all sensors have a bypass channel 608 over the flow channel 603. In some examples, the lid 640 includes bypass channels that travel around, to the side, or otherwise not above the flow channel. Further, some examples include additional channels to deliver fluids to particular sensors directly or indirectly.

FIG. 23 depicts an example of a sensor 710 with embedded light sources on its active surface 715. A cross sectional view of a sensor 710 having an active surface 715 is shown. The active surface 715 includes a plurality of reaction sites 790. Between the reaction sites 790 are interstitial regions that include light sources 760. In some examples, there is a one-to-one ratio between reaction sites and light sources. In other examples, there is less than a one-to-one ratio between reaction sites and light sources. In other examples, there is more than a one to one ratio between reaction sites and light sources. The active surface 715 of each sensor 710 may detect designated reactions simultaneously and/or in parallel.

FIG. 24 depicts an example of a portion of a flow cell with opposing sensors 710 with embedded light sources. Two sensors 710 are orientated facing each other, such that the active surface 715 of one sensor 710 faces the active surface 715 of the other sensor 710. A flow channel 703 is formed in the region between the active surfaces 715 of the sensors 710. Each active surface 715 of the sensor 710 includes both reaction sites 790 and light sources 760. The light sources 760 of the active surface 715 of one sensor 710, when activated, illuminate the reaction sites 790 of the active surface 715 of the other sensor 710. Likewise, the light sources 760 of the active surface of the other sensor 710, when activated, illuminate the reaction sites 790 of the active surface 715 of the one sensor 710. In some examples, the light sources 760 of each active surface 715 may be activated at the same time (simultaneously) thereby illuminating both active surfaces 715 of the opposing sensors 710 at the same time (simultaneously). In other examples, the light sources 760 of each active surface 715 may be activated at different times thereby illuminating the active surface 715 of one of the sensors 710 but not the other.

In some examples, all the light sources 760 of an active surface 715 of a sensor 710 may emit the same wavelength or wavelengths of light. In other examples, a subset of light sources 760 of an active surface 715 of a sensor 710 emit a subset of wavelengths of light, while a different subset of light sources 760 of an active surface 715 of a sensor 710 emit a different subset of wavelengths of light. By way of further example, a first sensor 710 may have an active surface 715 that includes a first set of light sources 760 that emit blue light, and a second set of light sources 760 that emit red light; a second sensor may have light sources that emit the same or different wavelengths than the first sensor.

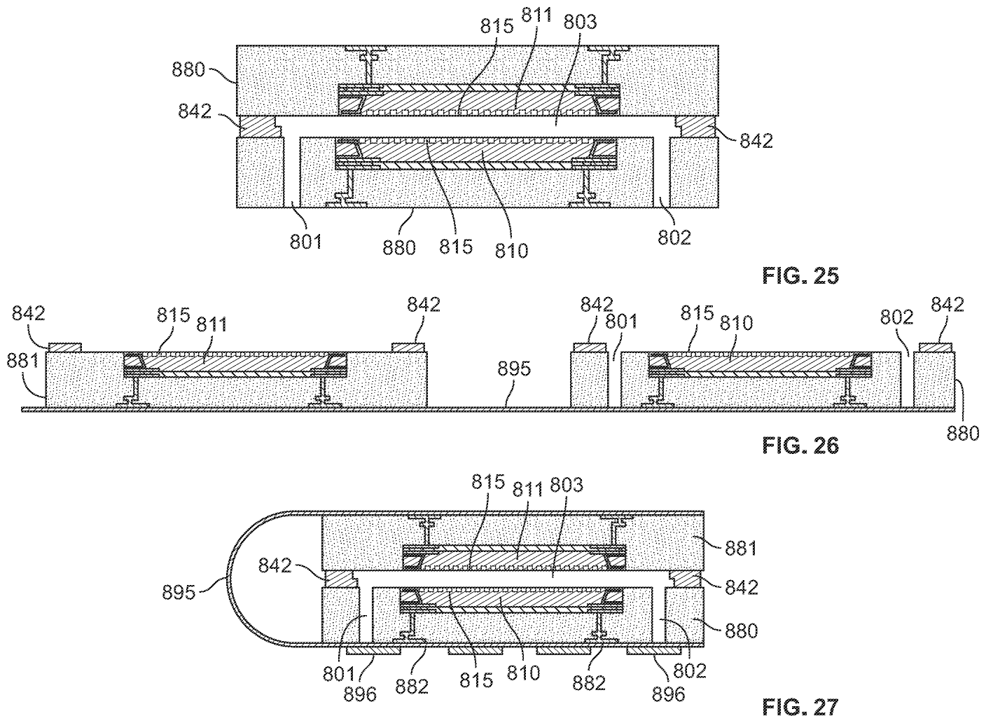

FIG. 25 depicts another example of a portion of a flow cell with opposing sensors with embedded light sources. A first sensor 810 having an active surface 815 resides within a mold 880. A second sensor 811 having an active surface 815 resides with a second mold 881. The active surface 815 of the first sensor 810 faces the active surface 815 of the second sensor 811. A flow channel 803 is formed in the region between the active surface 815 of the first sensor 810 and the active surface 815 of the second sensor 811. The mold 880 housing the first sensor 810 includes a fluid inlet 801 and a fluid outlet 802, each providing fluid access to the flow channel 803. In this example, the fluid inlet 801 and fluid outlet 802 each extend through the mold 880 on opposite sides of the sensor 810. Pillars 842 separate mold 840 and mold 841 which, in this example, corresponds to the distance between the active surface 815 of the first sensor 810 and the active surface 815 of the second sensor 811.

FIG. 26 depicts an example of sensors on a flexible surface. During the manufacturing process, in this example, a first sensor 810 and second sensor 811 are each coupled to a flexible surface 895. In some examples, the first sensor 810 and second sensor 811 are coupled to the flexible surface 985 using an adhesive. The first sensor 810 and second sensor 811 each have an active surface 815. The second sensor 811 resides in a mold 881. As shown in this figure, mold 881 has pillars 824 coupled thereto. The first sensor 810 resides in a mold 880. As shown in this figure, mold 880 has pillars 842 coupled thereto. The pillars 842 of the mold 880 mate with the pillars 842 of mold 881. In other examples, mold 881 has no pillars coupled thereto, but rather mates with pillars 842 coupled to the mold 880 of the opposing sensor. In other examples, mold 880 has no pillars coupled thereto, but rather mates with pillars 842 coupled to the mold 881 of the opposing sensor. In some examples, a pump (not shown in this figure) is coupled to the flow channel 803.

With continued reference to FIG. 26, the mold 880 includes a fluid inlet 801 and a fluid outlet 802. In some examples, the flexible surface 895 includes openings or apertures that provide fluid access through the flexible surface 895 to the fluid inlet 801 and fluid outlet 802. In other examples, the fluid inlet 801 and fluid outlet 802 are fluidically coupled to channels directly and not through the flexible surface 895. In other examples, the mold 880 does not include fluid inlet 801 and fluid outlet 802; rather, the fluid inlet 801 and fluid outlet 802 extend through or around pillars 842 that reside between the mold 880 and mold 881.

In some examples, the flexible surface may include standard flexible circuits made of polyimide films. The thickness of the flexible surface can vary, for example, from 10 μm to 100 μm. The flexible surface may also include copper electrical lines for electrically coupling the components attached thereto, including for example the sensors.

FIG. 27 depicts an example of sensors folded together on a flexible surface. Sensors placed on a flexible surface, such as that shown in FIG. 26, may be folded together such that the active surfaces of the sensors face each other, as shown in this FIG. 27. A first sensor 810 having an active surface 815 resides with in mold 880. A second sensor 811 having an active surface 815 resides with a second mold 881. The active surface 815 of the first sensor 810 faces the active surface 815 of the second sensor 811. A flow channel 803 is formed in the region between the active surface 815 of the first sensor 810 and the active surface 815 of the second sensor 811. The mold 880 housing the first sensor 810 includes a fluid inlet 801 and a fluid outlet 802, each providing fluid access to the flow channel 803. In this example, the fluid inlet 801 and fluid outlet 802 each extend through the mold 880 on opposite sides of the sensor 810. Pillars 842 separate mold 840 and mold 841 which, in this example, corresponds to the distance between the active surface 815 of the first sensor 810 and the active surface 815 of the second sensor 811. The molds 880 and 881 are each coupled to a flexible surface 895, for example, by an adhesive. The flexible surface 895 that resides between the molds 880 and 881 is able to flex and bend such that the active surface 815 of the first sensor 810 faces the active surface 815 of the second sensor 811. Electrical paths may extend through the flexible surface 895 from pads 882 on the bottom surface of the mold 880 to pads 896 on the opposing side of the flexible surface 895.

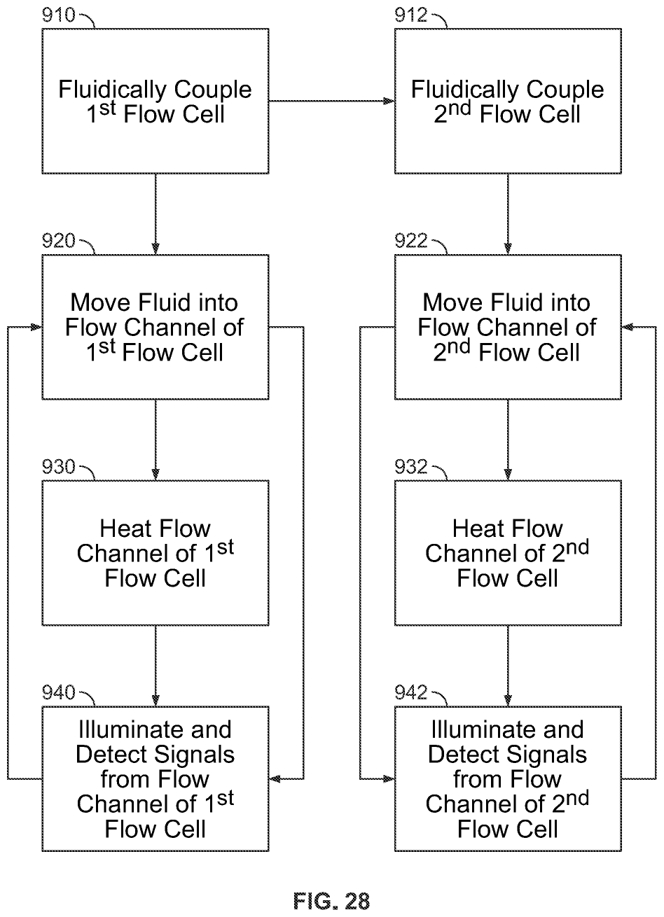

FIG. 28 depicts a flow chart of a method of operating an instrument with multiple individually addressable flow cells. A first flow cell is fluidically coupled to a reservoir 910. A second flow cell is fluidically coupled to a reservoir 912. In some examples, the first flow cell and second flow cell are fluidically coupled to a reservoir at or about the same time. In other examples, the first flow cell and second flow cell are fluidically coupled to a reservoir at different times, such as more than one minute apart. In other examples, only a first flow cell is fluidically coupled to a reservoir. In some examples, the first flow cell and second flow cell are coupled to the same reservoir. In other examples, the first flow cell and second flow cell are coupled to different reservoirs. In other examples, the first flow cell and second flow cell are coupled to multiple reservoirs. The reservoir or reservoirs may contain various reagents or washes.

After the first flow cell and/or second flow cell are fluidically coupled to a reservoir, fluid is moved from the reservoir into the flow channel of the first flow cell 920 and from the reservoir into the flow channel of the second flow cell 922. In some examples, the fluid from the reservoir is moved into the flow channel of the first flow cell 920 and second flow cell 922 at or about the same time. In other examples, the fluid from the reservoir is moved into the flow channel of the first flow cell 920 and second flow cell 922 at different times, such as more than one minute apart. The flow channel of the first flow cell is heated 930. The flow channel of the second flow cell is heated 932. In some examples, the flow channel of the first flow cell is heated 930 while the flow channel of the second flow cell is not, such that the fluid in the flow channel of the first flow cell is at a different temperature than the fluid in the flow channel of the second flow cell. In other examples, the flow channel of the first flow cell and the flow channel of the second flow cell are heated at or about the same time.

The flow channel of the first flow cell is illuminated, and signals detected/acquired 940, for example, by capturing an image of the flow channel or otherwise detecting emitted light from reaction sites on an active surface of a sensor of the flow cell. The flow channel of the second flow cell is illuminated and signals detected/acquired 942, for example, by capturing an image of the flow channel or otherwise detecting emitted light from reaction sites on an active surface of a sensor of the flow cell. In some examples, the flow channel of the first flow cell is illuminated, and signals detected/acquired 940 at or about the same time as the flow channel of the second flow cell is illuminated, and signals detected/acquired 942. In other examples, the flow channel of the first flow cell is illuminated, and signals detected/acquired 940 at a different time as the flow channel of the second flow cell is illuminated, and signals detected/acquired 942.

The process of moving fluid into the flow channel of a first flow cell and/or second flow cell 920 and 922, heating the flow channel of the first flow cell and/or second flow cell 930 and 932, and illuminating and detecting signals from the flow channel of the first flow cell and/or second flow cell 940 and 942 may form an iterative cycle of enzymatic manipulation and light or signal detection or acquisition. In some examples, the iterative cycle includes moving fluid into the flow channel of a first flow cell and/or second flow cell 920 and 922 and illuminating and detecting signals from the flow channel of the first flow cell and/or second flow cell 940 and 942, but not heating the flow channel of the first flow cell and/or second flow cell 930 and 932. A plurality of these iterative cycles may form a sequencing run, such as a DNA sequencing run. A sequencing run may occur on a single flow cell. Multiple sequencing runs may occur on multiple flow cells. In some examples, a sequencing run on a first flow cell starts at a different time than a sequencing run on a second flow cell. In some examples, each flow cell includes its own pump, whereby fluid may be moved from the reservoir and through the flow cell. In some examples, the flow cell includes logic circuitry and/or electronic memory and a processor to execute instructions stored on the electronic memory to actuate the pump on the flow cell. In further examples, the flow cell may include logic circuitry and/or electronic memory and a processor to execute instructions stored on the electronic memory to actuate one or more valves on an instrument to which the flow cell is removably coupled.

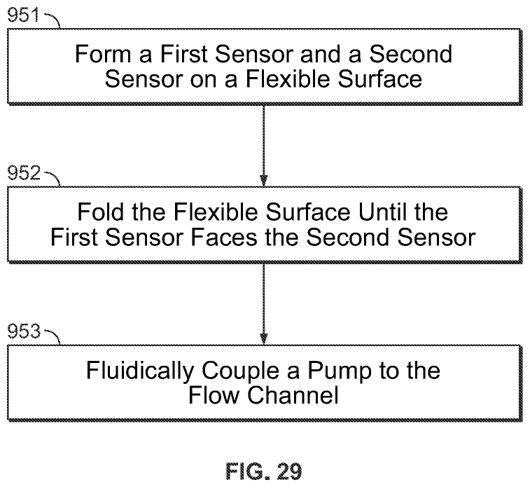

FIG. 29 depicts a flow chart of a method of making a flow cell with opposing sensors. In this example, the method includes forming a first sensor and a second sensor on a flexible surface 951, where each of the first and second sensors comprises an active surface having a plurality of reaction sites, where the active surface comprises a plurality of embedded illumination sources; and folding the flexible surface until the first sensor faces the second sensor 952, whereby a flow channel is formed between the first sensor and second sensor. The method may further include fluidically coupling a pump to the flow channel 953.

In some examples, a flow cell comprises a top layer with optically non-transparent or opaque features, including but not limited to, electrical components (e.g., electrodes) or physical structures (e.g., herringbone trenches). The integration of these performance enhancing features can help achieve faster SBS kinetics and positively impact the performance of the flow cells into which the top layer is integrated.

In some examples, the pillars are a single continuous material. In other examples, the pillars include multiple layers of materials. In other examples, the pillars comprise multiple components. In yet other examples, the pillars are an extension of and continuous with the mold.

The flowchart and block diagrams in the Figures illustrate the architecture, functionality, and operation of possible implementations of systems, methods, and computer program products according to various examples of the present implementation. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular examples only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, processes, operations, elements, and/or components, but do not preclude the presence or addition of one or more other features, integers, steps, processes, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more examples has been presented for purposes of illustration and description, but is not intended to be exhaustive or limited to in the form disclosed. Many modifications and variations will be apparent to those of ordinary skill in the art. Any example was chosen and described in order to best explain various aspects and the practical application, and to enable others of ordinary skill in the art to understand various examples with various modifications as are suited to the particular use contemplated.

It should be appreciated that all combinations of the foregoing concepts and additional concepts discussed in greater detail below (provided such concepts are not mutually inconsistent) are contemplated as being part of the subject matter disclosed herein at least to achieve the benefits as described herein. In particular, all combinations of claims subject matter appearing at the end of this disclosure are contemplated as being part of the subject matter disclosed herein. It should also be appreciated that terminology explicitly employed herein that also may appear in any disclosure incorporated by reference should be accorded a meaning most consistent with the particular concepts disclosed herein.

This written description uses examples to disclose the subject matter, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the above description is intended to be illustrative, and not restrictive. For example, the above-described examples (and/or aspects thereof) may be used in combination with each other. In addition, many modifications may be made to adapt a particular situation or material to the teachings of the various examples without departing from their scope. While the dimensions and types of materials described herein are intended to define the parameters of the various examples, they are by no means limiting and are merely provided by way of example. Many other examples will be apparent to those of skill in the art upon reviewing the above description. The scope of the various examples should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Forms of term “based on” herein encompass relationships where an element is partially based on as well as relationships where an element is entirely based on. Forms of the term “defined” encompass relationships where an element is partially defined as well as relationships where an element is entirely defined. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure. It is to be understood that not necessarily all such objects or advantages described above may be achieved in accordance with any particular example. Thus, for example, those skilled in the art will recognize that the systems and techniques described herein may be embodied or carried out in a manner that achieves or optimizes one advantage or group of advantages as taught herein without necessarily achieving other objects or advantages as may be taught or suggested herein.

While the subject matter has been described in detail in connection with only a limited number of examples, it should be readily understood that the subject matter is not limited to such disclosed examples. Rather, the subject matter can be modified to incorporate any number of variations, alterations, substitutions or equivalent arrangements not heretofore described, but which are commensurate with the spirit and scope of the subject matter. Additionally, while various examples of the subject matter have been described, it is to be understood that aspects of the disclosure may include only some of the described examples. Also, while some examples are described as having a certain number of elements it will be understood that the subject matter can be practiced with less than or greater than the certain number of elements. Accordingly, the subject matter is not to be seen as limited by the foregoing description, but is only limited by the scope of the appended claims.

1. An apparatus comprising:
a sensor with an active surface having a plurality of reaction sites, a lid, and a flow channel formed at least partially by the active surface of the sensor and the lid,
where the lid comprises an illumination source and a heater.

2. The apparatus of claim 1, wherein the sensor comprises a Complementary Metal-Oxide Semiconductor (CMOS) detection device comprising a plurality of detection pixels.

3. (canceled)

4. The apparatus of claim 1, wherein the lid further comprises at least one of a non-transparent material or an opaque material.

5. (canceled)

6. The apparatus of claim 1, wherein the lid further comprises a fluidic channel therein, where the fluidic channel is in fluidic communication with the flow channel.

7. The apparatus of claim 1, wherein the lid further comprises a reservoir and wherein the reservoir comprises at least one of a reagent or a buffer.

8. (canceled)

9. (canceled)

10. (canceled)

11. The apparatus of claim 1, wherein the heater is a resistive heater.

12. The apparatus of claim 1, wherein the lid is on an opposite side of the flow channel from the active surface of the sensor.

13. The apparatus of claim 1, wherein the illumination source comprises at least one of a light emitting diode (LED), a plurality of LEDs, a thin film organic LED, or a silicon-based LED.

14. (canceled)

15. The apparatus of claim 1, wherein the illumination source is located along a periphery of the lid and wherein the lid comprises a plurality of light guides, whereby the light guides guide light from the illumination source toward the active surface of the sensor.

16. (canceled)

17. (canceled)

18. (canceled)

19. The apparatus of claim 1, wherein the illumination source is on a bottom surface of the lid, where the bottom surface of the lid faces the active surface of the sensor.

20. (canceled)

21. (canceled)

22. (canceled)

23. (canceled)

24. (canceled)

25. A method comprising:
fluidically coupling a first flow cell and a second flow cell to a reservoir, wherein the first flow cell and second flow cell each comprise a sensor with an active surface having a plurality of reaction sites, a lid, a heater, and a pump, where the lid and the sensor at least partially form a flow channel, where the pump is in fluidic communication with the flow channel;
moving fluid from the reservoir into the flow channel of the first flow cell using the pump of the first flow cell and fluid from the reservoir into the flow channel of the second flow cell using the pump of the second flow cell; and
heating fluid in the flow channel of the first flow cell using the heater of the first flow cell such that fluid in the flow channel of the first flow cell is at a different temperature than the fluid in the flow channel of the second flow cell.

26. The method of claim 25, wherein moving fluid from the reservoir into the flow channel of the first flow cell does not occur while moving fluid from the reservoir into the flow channel of the second flow cell.

27. The method of claim 25, wherein the reservoir comprises at least one of a reagent or a buffer.

28. (canceled)

29. The method of claim 25, further comprising illuminating at least a portion of the reaction sites of the sensor of the first flow cell or illuminating at least a portion of the reaction sites of the sensor of the second flow cell.

30. (canceled)

31. The method of claim 29, wherein illuminating at least a portion of the reaction sites of the sensor of the second flow cell does not occur while illuminating at least a portion of the reaction sites of the sensor of the first flow cell.

32. The method of claim 29, wherein an illumination source in the lid of the first flow cell illuminates at least a portion of the reaction sites of the sensor of the first flow cell and an illumination source in the lid of the second flow cell illuminates at least a portion of the reaction sites of the sensor of the second flow cell.

33. (canceled)

34. The method of claim 25, wherein a first sequencing run is performed on the first flow cell, and a second sequencing run is performed on the second flow cell, where the first sequencing run and second sequencing run start at different times.

35. A device comprising:
a sensor with an active surface having a plurality of reaction sites, a lid, and a flow channel formed at least partially by the active surface of the sensor and the lid,
where the lid comprises a heater.

36. The apparatus of claim 35, wherein the heater is a resistive heater.

37. The apparatus of claim 35, wherein the sensor comprises a Complementary Metal-Oxide Semiconductor (CMOS) detection device comprising a plurality of detection pixels.

38. (canceled)

39. The apparatus of claim 35, further comprising a pump, where the pump is fluidically coupled to the flow channel.

40. (canceled)

41. The apparatus of claim 39, wherein the lid further comprises an outlet port, wherein the pump is adjacent to the outlet port of the lid.

42. The apparatus of claim 39, wherein there is no removable connection between the flow channel and the pump.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)
